SEQUENCE LISTING (13 sequences)

SEQ ID NO: 1 - DNA, 5220 bp, artificial sequence
  nucleotide sequence for mouse TEM8
cggaactgcttcgactgcaaagcttcaagcgcagcctgggagcggcctggtggccctatcccggcagctccacacagcagaacgccctgggtccctgaaactcgaaacccgggctcagaaccagcggaaaccaaagcgaaatccttgaacttctctgaacaattgcttccgggcgtttgctgagagccgggggacctgaccggagcccaggccgcgtatggcgcgcccctgatgtcacacggacgccagcgaggccagcgctccggctgcagcatggaccgcgcggggcgcctgggtgcgggcctgcggggactctgcgtggctgcactcgtgctcgtgtgcgccggacacgggggccgccgcgaggatgggggaccagcttgctacggaggattcgacctctacttcatcctggacaagtcaggaagtgtgctgcaccactggaatgaaatctactacttcgtggagcagttggctcatagattcatcagcccacagctaaggatgtccttcattgtcttctctactcgagggacaactttaatgaaactaactgaggacagggaacagatccgacaaggcctagaagagctccagaaagttctgccaggaggagacacttacatgcacgaaggattcgagagggccagtgagcagatttactatgagaacagtcaaggatacaggacggcgagcgtcatcatcgcgttgacggatggggagctgcacgaggacctcttcttctactcagagagggaggctaaccgatcccgagaccttggtgcgattgtttactgcgttggcgtgaaggatttcaatgaaactcagttggctcggattgcagacagtaaggaccacgtgtttcctgtgaacgacggcttccaggctctccaaggcattatccactcaattttaaagaaatcctgcatcgaaattctggcggctgaaccatccaccatctgcgcgggagagtcctttcaagtggtcgtaagaggaaatggcttccgacatgcccgcaatgtggacagggtcctctgcagcttcaaaatcaatgactcagtcacgctcaatgagaagccctttgctgtggaagacacttatttgctgtgcccagcaccaatcttgaaagaagttggcatgaaagctgcactgcaggtcagcatgaacgacggcctgtccttcatctccagttctgtcatcatcaccaccacacactgttcagacggctccatcctggcgattgctctgctggtcctcttcctgctgctggccctggcgctgctctggtggttctggcccctctgctgcacagtgatcatcaaggaggtccctccaccccctgttgaggagagtgaggaagaagacgatgatggtttgccaaagaagaaatggcccacagtagatgcctcttattatggtggacgcggtgtgggaggcattaaaagaatggaggtccgctggggagaaaagggctccacagaagaaggggcgaagttagaaaaggcaaagaatgcacgagtcaagatgccagagcaagaatatgagttcccagaaccccgaaacctcaacaacaacatgcgccggccttcctcgcctcggaagtggtactcgcccatcaagggaaaactcgatgccttgtgggttctgctgagaaaaggatatgaccgagtgtctgtgatgaggccacagccaggagacacgggacgctgtatcaacttcaccagagtgaagaacagtcagccagccaagtatcccctgaacaacacctaccaccccagctccccacctcccgctcctatctacacacccccaccccctgctccccactgccctcccccagcccccagtgcccccactcctcccattccttccccaccatccactctcccccctcctcctcaggccccaccccctaacagggcacctcccccctcccgacctcctccaaggccttctgtctagaacccaaagtccgagctctgggctgcctgagcaactccagcaggaggcttctctgctgaaagaaagatctgcccagcctatgtggtgagtggcggctgatgtttgcacgatttaaaagcaagtcgtgatgggcagaacaaaatgggcattttgaactgcctgaagacagacaatgagacaataacagtcacattatagcctgtgacccctcacctctagaggaaggttcccgagatggccacattgccacagtgctctcagccagattatgtcccatgaagaccaggaagaaagtgacttccaagaatggaatgcagcattggataagaaacacctggctgagattctgacctcactgatttgactcttgattcttggactgggagccaggccatctccacccctggtaccacccagcaactctgaaaatgtgcagtgtccctagtatgcatcgaataggtatccaactgggatctgcaggttgccttataaagagcatatgctctattctctttcccgaacttcctggtttcccagtgatgagggaaggggaaaggtgttgccatgcttagaagttagaggacgtcagtgctcagcactgatggagaagcgttgatgggagtgtccagctcttacatctagaaatggctggcttcagcaggcacagttcctaaaccaacaagccttgtcattgtcaaaggcaacctactaatgattcaccttaaacatcaaggttgactgtggcataggtcagagctgatcacacagaaccttccccatgaaatcgcaaggttcctcatcttcaaatacccaggaccccagagatttctaaatccagctaagagacagtagtcctgacttggcaagaaaaccattcccagttgttttactctgaaacaggccgttgtatgtatggtatatctctccttggcctttcaacctgctcacaagtattaccagttatgaagcaaggagaaatacatccagtgtgtaatagaaaagctctgcccacaatccccatgtcactcctctacattattctgaagctgcttggtcagtgagccctttaacctcatgtagactctggacactgtcacccaatcatgaaaacagaggtcattgtcaaaggcagtgtatagcctgtacaaaaatgatgcttccttcctcagtttccacaggccccaaaattcctgtcttaggctcctaaacctctaaactttttcctggaacaaaagatataaaacgggcataagtttttatgttttgggctgtgatctccaaagatccttcaagaactcaagttagcctcattcttccagcttgtttagaacagaggcatccaggtgtcatgcactccatagacaccaatccttgttcccaaggcagacattattaatcaatctcagcactagttctcaatttaatccaattatatttttccacagtacttcacatctcttatgacctgttggtcatcagttagaattgagagagataaacactgtttgtaatccctaccttagaaagaaaagcagaggagaatgggggaaccaccagcataaaagttattatctggggaaaatcgacctgaaagaacgcccaagtccaagacctatggtgctgacaccaaagtaacactttcccaagtgtaccccagaccccactcttctccctgtggccaccactccctgcttttcaggagttgtgaaaaagatctccttcacccttactgtgcccccatattagaacaaggcttgtttagtgtagtccttgttaaacaggtgccagaatgtctcagccacctgagatgacattgctgggccccagaaaaccattccaaggagaatgggctccccaggctcagagcatgcaactatgagcccatggcaactgttttgactgctggcagtacaaaacgggccaccccacattacagctgcaggatttgtgcagccataagaaagtatgaaccaagatgctggtgttgctgttcaacaagcatgggcttcggggaaggcagcagactccgagagcaggccttgtgcagtgtcccaaggggctgtggtgaagtgtctgaggaaaaatgaatgctgatacatggtgattctgagaagaatttgcaaggtttgaccttagaatttatggaatgtcttccctggtcattcagaattatggctagaagtttctagaaaccgtcaaggttaatacctttcagagtaggtgattacaggcaggaagagctttgatgtggtttacaaagcccatcagttctgtgtcattccctgtaagcaacaggagatggtggttgtgattagcaaactgcatgtgttatttgtttgactccttgttattgtccttacggaggattttttttatataagccaaattttgttgtatatattcatattccacgtgacagatggaagcacgtcctatcagtgtgaataaaaagaacagttgtagtaaattattaaagccagtgatttcatggcaggttaccctaccaagctgtgcttgttgatctcccatgaccatactgcttttacaatgtacaaatagttcctaggtgacgagaccctcctttacataatgccgatgacagccttgctgggaactgcggtccttctgctgtgacagccagctcgaaaacaggtcctgcctggagcttgccacacactttagggagacataagagctgtctttccccagcgtcagggacaaagctaccataaagaagtggaaaagtcttggctctccagcctgggacagaggtctctctggaaccccaaggaagagcagaaatgatccttgcctgccactgcacacaatgtgatggtggaaaatccatcaaggaataattgtgagataatgaccgacagttcaggcgcaaagggaattcatgctgtgtaaagtgggtggaattcgtttgcaagctatgcaaagcctgatcttactcaccaggaggatggaaagggtttttttagttatctgagctcagctgagttatcacgcttggagaaccgatttaaaggaattagaatatgatttctgaatacacataacattaaactcttctctttttctatggtaatttagttatggacgttcagcgtctctgagttattgttataaaagacttgtcatcaccgcactgtgctgtaggagactgggctgaacctgtacaatggtataccctggaagttgcttttttaaaaaaaaataataataaacacctaaaatcaaaaaaaaaaaaaaaaa

SEQ ID NO: 2 - protein, 561 aa, artificial sequence
  amino acid sequence for mouse TEM8
MDRAGRLGAGLRGLCVAALVLVCAGHGGRREDGGPACYGGFDLYFILDKSGSVLHHWNEIYYFVEQLAHRFISPQLRMSFIVFSTRGTTLMKLTEDREQIRQGLEELQKVLPGGDTYMHEGFERASEQIYYENSQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFAVEDTYLLCPAPILKEVGMKAALQVSMNDGLSFISSSVIITTTHCSDGSILAIALLVLFLLLALALLWWFWPLCCTVIIKEVPPPPVEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTRCINFTRVKNSQPAKYPLNNTYHPSSPPPAPIYTPPPPAPHCPPPAPSAPTPPIPSPPSTLPPPPQAPPPNRAPPPSRPPPRPSV

SEQ ID NO: 3 - protein, 252 aa, artificial sequence
  amino acids 27-279 for mouse TEM8
GRREDGGPACYGGFDLYFILDKSGSVLHHWNEIYYFVEQLAHRFISPQLRMSFIVFSTRGTTLMKLTEDREQIRQGLEELQKVLPGGDTYMHEGFERASEQIYYENSQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFAVEDTYLLC

SEQ ID NO: 4 - DNA, 5540 bp, artificial sequence
  nucleotide sequence for human TEM8
aattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattctaaaaattggttggcaatgccagtataccaacaatcatgatcagctgaaagaaacagatattttaaattgccagaaaacaaatgatgaggcaactacagtcagatttatagccagccatctatcacctctagaaggttccagagacagtgaaactgcaagatgctctcaacaggattatgtctcatggagaccagtaagaaaatcatttatctgaaggtgaaatgcagagttggataagaaatacattgctgggtttctaaaatgctgccttcctgcctctactccacctccatccctggactttggacccttggcctaggagcctaaggaccttcacccctgtgcaccacccaagaaagaggaaaactttgcctacaactttggaaatgctggggtccctggtgtggtaagaaactcaacatcagacgggtatgcagaaggatgttcttctgggatttgcaggtacataaaaaatgtatggcatcttttccttgcaaattcttccagtttccaagtgagaaggggagcaggtgtttactgatggaaaaggtatgttgctatgttgatgtgtaagtgaaatcagttgtgtgcaatagacaggggcgtattcatgggagcatcagccagtttctaaaacccacaggccatcagcagctagaggtggctggctttggccagacatggaccctaaatcaacagacaatggcattgtcgaagagcaacctgttaatgaatcatgttaaaaatcaaggtttggcttcagtttaaatcacttgaggtatgaagtttatcctgttttccagagataaacataagttgatcttcccaaaataccatcattaggacctatcacacaatatcactagttttttttgtttgtttgttttttgttttttttcttggtaaagccatgcaccacagacttctgggcagagctgagagacaatggtcctgacataataaggatctttgattaacccccataaggcatgtgtgtgtatacaaatatacttctctttggcttttcgacatagaacctcagctgttaaccaaggggaaatacatcagatctgcaacacagaaatgctctgcctgaaatttccaccatgcctaggactcaccccatttatccaggtctttctggatctgtttaatcaataagccctataatcacttgctaaacactgggcttcatcacccagggataaaaacagagatcattgtcttggacctcctgcatcagcctattcaaaattatctctctctctagctttccacaaatcctaaaattcctgtcccaagccacccaaattctcagatcttttctggaacaaggcagaatataaaataaatatacatttagtggcttgggctatggtctccaaagatccttcaaaaatacatcaagccagcttcattcactcactttacttagaacagagatataagggcctgggatgcatttattttatcaataccaatttttgtggccatggcagacattgctaatcaatcacagcactatttcctattaagcccactgatttcttcacaatccttctcaaattacaattccaaagagccgccactcaacagtcagatgaacccaacagtcagatgagagaaatgaaccctacttgctatctctatcttagaaagcaaaaacaaacaggagtttccagggagaatgggaaagccagggggcataaaaggtacagtcaggggaaaatagatctaggcagagtgccttagtcagggaccacgggcgctgaatctgcagtgccaacaccaaactgacacatctccaggtgtacctccaaccctagccttctcccacagctgcctacaacagagtctcccagccttctcagagagctaaaaccagaaatttccagactcatgaaagcaaccccccagcctctccccaaccctgccgcattgtctaatttttagaacactaggcttcttctttcatgtagttcctcataagcaggggccagaatatctcagccacctgcagtgacattgctggacccctgaaaaccattccataggagaatgggttccccaggctcacagtgtagagacattgagcccatcacaactgttttgactgctggcagtctaaaacagtccacccaccccatggcactgccgcgtgattcccgcggccattcagaagttcaagccgagatgctgacgttgctgagcaacgagatggtgagcatcagtgcaaatgcaccattcagcacatcagtcatatgcccagtgcagttacaagatgttgtttcggcaaagcattttgatggaatagggaactgcaaatgtatgatgattttgaaaaggctcagcaggatttgttcttaaaccgactcagtgtgtcatccccggttatttagaattacagttaagaaggagaaacttctataagactgtatgaacaaggtgatatcttcatagtgggctattacaggcaggaaaatgttttaactggtttacaaaatccatcaatacttgtgtcattccctgtaaaaggcaggagacatgtgattatgatcaggaaactgcacaaaattattgttttcagcccccgtgttattgtccttttgaactgttttttttttattaaagccaaatttgtgttgtatatattcgtattccatgtgttagatggaagcatttcctatccagtgtgaataaaaagaacagttgtagtaaattattataaagccgatgatatttcatggcaggttattctaccaagctgtgcttgttggtttttcccatgactgtattgcttttataaatgtacaaatagttactgaaatgacgagacccttgtttgcacagcattaataagaaccttgataagaaccatattctgttgacagccagctcacagtttcttgcctgaagcttggtgcaccctccagtgagacacaagatctctcttttaccaaagttgagaacagagctggtggattaattaatagtcttcgatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctataggagactgggcaaaacctgtacaatgacaaccctggaagttgctttttttaaaaaaataataaatttcttaaatcaaaaaaaaaaaaaaaaaaaa

SEQ ID NO: 5 - protein, 564 aa, artificial sequence
  amino acid sequence for human TEM8
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 6 - DNA, 24 bp, artificial sequence
  Sense strand oligonucleotide sequence to make pVAXXCDneu.
cgcaagcttcatcatggagctggc

SEQ ID NO: 7 - DNA, 24 bp, artificial sequence
  Antisense strand oligonucleotide sequence to make pVAXXCDneu.
gcagaattcttatgtcaccgggct

SEQ ID NO: 8 - DNA, 25 bp, artificial sequence
  Sense strand oligonucleotide sequence to amplify TEM8 cDNA.
ggactctgcgtggctgcactcgtgc

SEQ ID NO: 9 - DNA, 25 bp, artificial sequence
  Antisense strand oligonucleotide sequence to amplify TEM8 cDNA.
agagcagcgccagggccagcagcag

SEQ ID NO: 10 - DNA, 36 bp, artificial sequence
  FWKpnIm8 sense strand oligonucleotide sequence to clone 28-278 amino acid sequence of TEM8.
gggggtaccgcaatgggccgccgcgaggatggggga

SEQ ID NO: 11 - DNA, 34 bp, artificial sequence
  RVEcoRIm8 antisense strand oligonucleotide sequence to clone 28-278 amino acid of TEM8.
ggtggaattcctagcacagcaaataagtgtcttc

SEQ ID NO: 12 - DNA, 55 bp, artificial sequence
  attB1bis oligonucleotide sequence to amplify TEM8 recombinant protein.
ggggacaagtttgtacaaaaaagcaggcttgatgggccgccgcgaggatggggga

SEQ ID NO: 13 - DNA, 51 bp, artificial sequence
  attB2bis oligonucleotide sequence to amplify TEM8 recombinant protein.
ggggaccactttgtacaagaaagctgggtcgcacagcaaataagtgtcttc

(19) United States
(12) Patent Application Publication
(10) Pub. No.: US 2008/0171058 A1
(43) Pub. Date: Jul. 17, 2008

(54) Compositions and Methods For Synergistic Induction of Antitumor Immunity

(76) Inventors: Polly Gregor, New York, NY (US); Alan Houghton, New York, NY (US); Franco M. Venanzi, Camerino (IT); Antonio Concetti, Falerone (IT)

Correspondence Address: Benjamin Aaron Adler;Adler & Associates, 8011 Candle Lane, Houston, TX, 77071, US

(21) Appl. No.: 10/579,356

(22) PCT Filed: Nov. 15, 2004
(86) PCT No.: PCT/US2004/038022

§ 371 Date: Dec. 3, 2007

Related U.S. Application Data
(60) Provisional application No. 60/519,498, filed on Nov. 13, 2003.

(51) Int. Cl. A61K 9/127; A61K 39/00; A61K 39/02; A61P 35/04

(52) U.S. Cl. 424/185.1; 424/277.1; 424/235.1; 424/450

(57) ABSTRACT

The present invention discloses a synergistic effect between vaccines encoding tumor-associated antigen and vaccines encoding tumor endothelial marker (8) (TEM8). Potent antitumor immunity was generated by the combined use of these vaccines. Tumor supported by vasculature and for which a tumor-associated antigen has been defined can be treated with this approach.

### BACKGROUND OF TE INVENTION

1. Field of the Invention

The present invention relates generally to the field of antitumor immunity. More specifically, the present invention relates to compositions and methods of inducing antitumor immune responses by targeting both tumor-associated antigen(s) and tumor endothelia.

2. Description of the Related Art

Many of the anticancer gene therapy trials are actually based on immunological approaches where the aim is to establish a permanent systemic immune response like that provided by a vaccine. The interest in cancer vaccines was rekindled when it was clear that immunogenic tumor-associated antigens do exist as revealed by the presence of B-cell and T-cell immune responses to those antigens in some patients.

One of the well-known tumor-associated antigens is the HER2/neu oncogene product, the transmembrane protein p185. The p185 protein is a tyrosine kinase receptor involved in cell growth as well as carcinogenesis. Over-expression of p185 has been observed in many human cancers such as breast, ovarian, uterine, stomach, prostate, and lung cancer. The DNA sequence coding for the extracellular domain of p185 has been inserted into a plasmid and recently used in clinical immunotherapy.

Nevertheless, only a few tumor antigens have been chosen as immunotherapeutic targets, and despite several significant attempts, genetic immunotherapy has not yet yielded the anticipated clinical results. One of the reasons for the lack of success lies in the use of mono-therapeutic targets, which appear to be unable to effectively halt the development of cancer. It is known that tumor cells are genetically unstable, which means that tumor development is accompanied by a high mutation rate. Consequently, immune responses elicited toward a certain antigen may no longer be effective as mutations arise and the immune system fails to recognize a mutant form of the antigen. In order to overcome these problems, a new generation of vaccines is currently in development which combines many epitopes in the same vaccine or are directed to multiple targets.

The growth of solid tumors beyond micrometastasis requires the generation of an independent blood supply. The recruitment of host endothelium into tumor vasculature is thus believed to play a key role in tumor progression and metastasis. For this reason, molecules expressed in tumor vasculature are being developed as targets for chemotherapy and immunotherapy.

Angiogenesis associated with physiological and pathological conditions utilizes a partially overlapping set of molecules. In physiological neovascularization, such as embryogenesis, corpus luteum and wound-healing, angiogenic and antiangiogenic molecules are released by accessory cells. These factors control the migration and proliferation of endothelial cells, their morphogenetic (capillary) differentiation and the concurrent remodeling of the stromal matrix. While these processes are tightly regulated in physiological conditions, a persistent deregulated angiogenesis is observed in cancer as well as in other diseases. The endothelial cells themselves, however, are not transformed, and therefore are more susceptible to regulation than are tumor cells. Inhibition of tumor growth by attacking the tumor's vasculature offers a primary target for antiangiogenetic intervention. Indeed, preclinical animal models developed with endostatin, angiostatin, VEGF antagonists, and many other new generation angiogenesis inhibitors convincingly validated the guiding principles of this concept and led to clinical trials. However, despite considerable promise shown by these agents in pre-clinical studies, no significant durable clinical responses have been observed when compared with standard cyto-reductive modalities that target tumor cells.

It has been reported that vaccines comprised of xenogeneic (human) endothelial cells (HUVEC) induce anti-tumor immunity in a mouse model (Wey et al., 2000). More recently, human umbilical vein endothelial cell vaccines were examined in a transgenic (HER-2/neu+) mouse model of breast cancer. HER2/neu transgenic mice were immunized with a combination of xenogeneic human umbilical vein endothelial cells and a HER-2/neu DNA vaccine give by intra-muscular injection (Venanzi et al., 2002). In these experiments, while the separate immunological stimuli showed either limited (neu DNA vaccine) or no (human umbilical vein endothelial cell) tumor protection, mice receiving the combination were protected from aggressive breast cancer. This protection against mammary carcinogenesis shows proof of principle that combining immune responses against both cancer cells and tumor endothelia may prove to be an important advance in the design of tumor vaccines.

Recently, Carson-Walter et al. (2001) disclose the presence of at least 46 transcripts that are named tumor endothelial markers (TEMs) which are specifically elevated in tumor-associated endothelium. Tumor endothelial markers include integrins, additional growth factor receptors, as well as other molecules involved in downstream signaling events. The expression of tumor endothelial markers is clearly detectable in the vasculature of tumors in both human and mice.

The expression pattern of TEM8 is especially intriguing because it suggests that this gene may be highly specific to tumor angiogenesis and not required for normal adult angiogenesis. Both the human and mouse TEM8 protein possess large cytoplasmic tails which share at least seven potential phosphorylation sites, supporting the hypothesis that TEM8 is involved in transducing extracellular signals into the cells.

While it is theoretically possible to immunize human patients with murine endothelial cell lines, for regulatory reasons, it is far more practical to identify individual molecules that can substitute for the human umbilical vein endothelial cells in clinical grade vaccines. One very promising alternative to xenogeneic human umbilical vein endothelial cells is using the tumor endothelial marker 8 (TEM8) antigen to enhance the antitumor effects of tumor-specific DNA vaccines such as HER2/neu.

There is a need for, and the prior art is deficient in, approaches that enhance antitumor immunity separately induced by targeting tumor-associated antigens or molecules involved in tumor angiogenesis. The present invention fulfills this long-standing need and desire in the art by providing compositions and methods of targeting both tumor-associated antigens and the TEM8 molecule in the same vaccine.

### SUMMARY OF THE INVENTION

The present invention reports a surprising finding that the products of the tumor endothelial marker 8 gene (TEM8), when used as an immunogen, is able to boost the immune response towards tumor-associated antigens, thus allowing effective and long lasting immunoprotection from tumor development.

Tumor endothelial marker 8 (TEM8) is expressed in tumor neovasculature, fetal liver and brain, but not in normal adult tissues. The anthrax toxin receptor (ATR) is a splice variant of TEM8 and is identical in amino acid sequence throughout the extracellular and transmembrane domains.

Initial immunization experiments were performed using the extracellular domain of TEM8/ATRex and HER2/neu in a transgenic mouse model of breast cancer. Mice transgenic for the rat neu proto-oncogene were immunized by intramuscular injection three times at bi-weekly intervals with 100 mg DNA encoding TEM8/ATRex and with DNA encoding the extracellular domain of rat neu. Mice were challenged with a syngeneic tumor line derived from the FVB/neu mouse strain. In this experiment, mice immunized with TEM8/ATRex alone showed no protection from tumor growth, whereas immunization with HER2neu gave partial protection. In contrast, immunization with HER2/neu plus TEM8 gave nearly complete protection for over 65 days (FIG. 4).

In order to demonstrate that the effects of TEM8 vaccines increase immunity to a range of tumors, the investigators immunized mice with TEM8 plus human tyrosinase-related protein 1 (hgp75), a melanoma differentiation antigen. In initial experiments, all mice received 5 weekly injections of 4 mg (1 mg in each quadrant of the abdomen) of pINGTEM8 DNA by particle bombardment. Each week, three days following the pINGTEM8 injections, mice were immunized with 4 mg (1 mg in each quadrant of the abdomen) of hgp75 DNA by particle bombardment. Five days following the last DNA injection of hgp75, the immunized mice were challenged with B16 tumor cells intradermally. Tumors were measured with calipers every 2-3 days for a minimum of 2 months.

As in the case with HER2/neu immunization and FVB/neu tumor challenge, pINGTEM8 vaccination alone had no effect on B16 tumor growth. As shown in FIG. 5, at day 40 following tumor challenge, hgp75 immunization alone afforded 57% tumor protection, while hgp75+pINGTEM8 showed 87% tumor free survival. The effect is thus synergistic and not additive.

Further, the present invention also investigated whether the immunity induced by hgp75 depended on TEM8. As shown in FIG. 6, immunization with either TEM8 DNA, PSMA DNA alone or both PSMA DNA and hgp75 DNA did not provide immunity against the B16 tumor. However, immunization with both TEM8 DNA and hgp75 DNA provided maximum protection against B16 tumor. This demonstrated that immunity induced by hgp75 depended on TEM8. Additionally, as shown in FIG. 7, the synergistic effect of TEM8 with hgp75 in inducing antitumor effect was completely lost in mice lacking CD8+ T cells. This demonstrated that CD8+ T cells mediated TEM8 induced immunity. Further as shown in FIG. 8, it was observed that TEM8 also increased tumor immunity in a surgical resection model of B16 Melanoma.

It is an object of the present invention to provide a preparation comprising nucleic acid molecules encoding a tumor-associated antigen(s) and a TEM8 gene product, or their immunogenic fragments or derivatives, to be used simultaneously, separately or sequentially for preventive or therapeutic treatment of cancer. Any tumor supported by vasculature and for which a tumor-associated antigen, such as a differentiation antigen, has been defined can be treated with this approach.

The present invention is directed to compositions useful as a vaccine and methods of using these compositions to induce antitumor immunity. In one embodiment of the present invention, there is provided a composition useful as a vaccine. This composition comprises a vector comprising nucleic acid sequence encoding a tumor associated antigen or a fragment thereof and a vector comprising nucleic acid sequence encoding a tumor endothelial marker 8 or a fragment thereof and a pharmaceutically acceptable carrier.

In another embodiment of the present invention, there is a related composition useful as a vaccine. This composition comprises a vector comprising nucleic acid sequence encoding a tumor-associated antigen or a fragment thereof, nucleic acid sequence encoding a tumor endothelial marker 8 or a fragment thereof and a pharmaceutically acceptable carrier.

In yet another embodiment of the present invention, there is another related composition useful as a vaccine. This composition comprises a vector comprising nucleic acid sequence encoding a tumor associated antigen or a fragment thereof, a recombinant protein comprising tumor endothelial marker 8 or a fragment thereof and a pharmaceutically acceptable carrier.

In further yet another embodiment of the present invention, there is yet another related composition useful as a vaccine. This composition comprises a recombinant protein comprising a tumor associated antigen or a fragment thereof, a recombinant protein comprising a tumor endothelial marker 8 or a fragment thereof and a pharmaceutically acceptable carrier.

In still yet another embodiment of the present invention, there is another related composition useful as a vaccine. This composition comprises a recombinant protein comprising a tumor associated antigen or a fragment thereof, a vector comprising nucleic acid sequence encoding a tumor endothelial marker 8 or a fragment thereof and a pharmaceutically acceptable carrier.

Additionally, in other embodiments of the present invention, there are provided methods of using these claimed vaccine compositions to induce antitumor immunity in human.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

FIG. 1 is a plasmid map of pECDrneu.

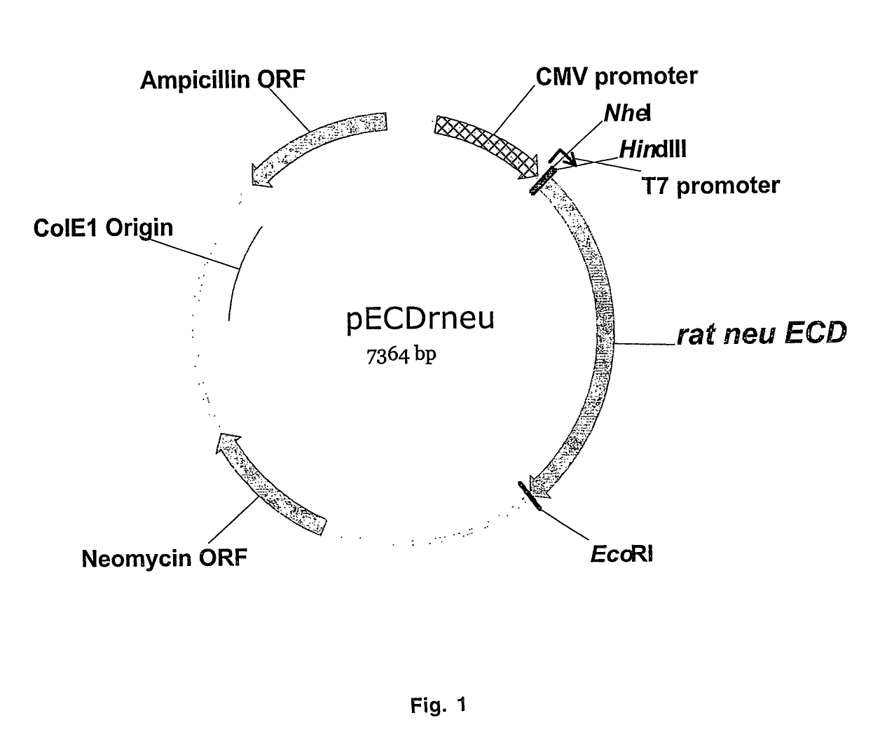

FIG. 2A is a plasmid map of pcDNA3TEM8.

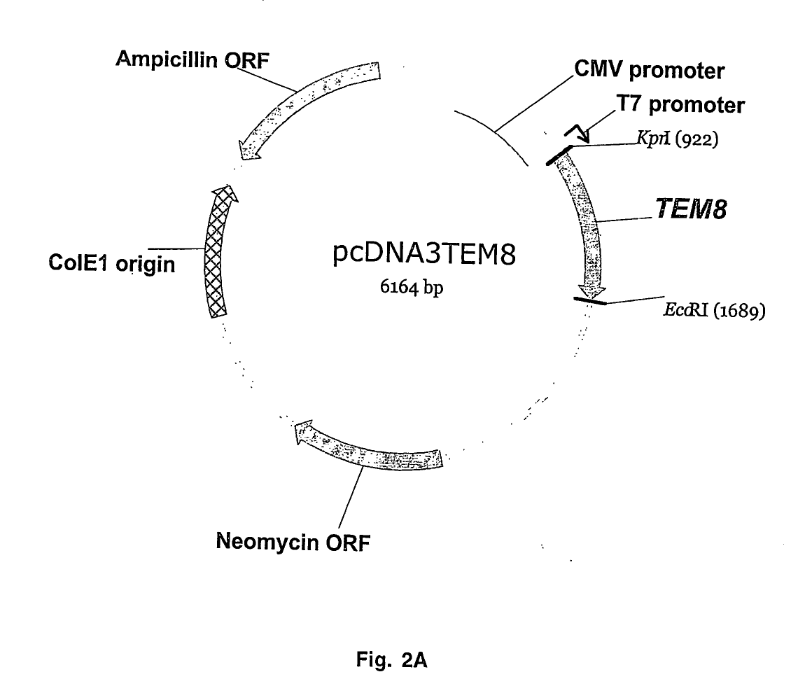

FIG. 2B is a plasmid map of pINGTEM8.

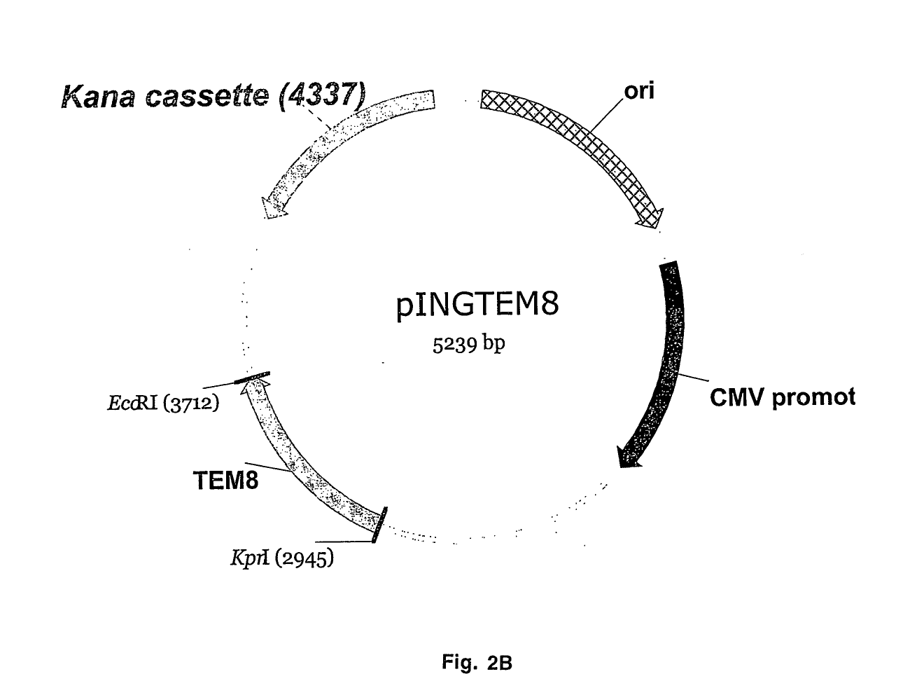

FIG. 3 shows Northern blot analysis of TEM8 mRNA expression in spontaneous mammary tumors in FVB/neu transgenic mice. RNA from healthy (N) and tumor-affected (T) mammary glands were probed with anti-sense (T7) or sense (SP6) TEM8/ATR probes or with actin riboprobes.

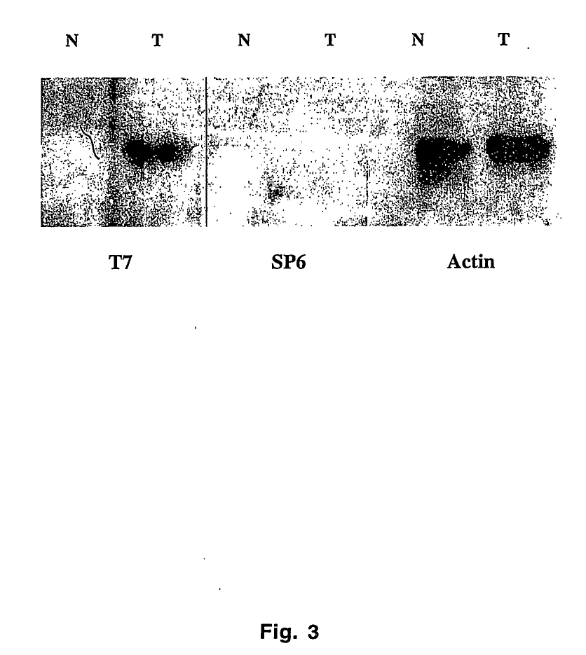

FIG. 4 shows mammary tumor development in control non-immunized mice or mice immunized with HER2/neu DNA vaccine (nex), pcDNA3TEM8 DNA vaccine (TEM8), or both HER2/neu and TEM8 DNA vaccines (nex+TEM8).

FIG. 5 shows melanoma development in control non-immunized mice or mice immunized with hgp75 DNA vaccine, pINGTEM8 DNA vaccine, or both hgp75 and pINGTEM8 DNA vaccines.

FIG. 6 shows that TEM8 and not PSMA improves immunity induced by hgp75. Mice were not immunized (no treatment) or immunized with hgp75 DNA vaccine, PSMA (dummy protein) DNA vaccine, both PSMA and hgp75 DNA vaccines, pINGTEM8 DNA vaccine alone or both pINGTEM8 and hgp75 DNA vaccines.

FIG. 7 shows the role of CD8⁺ T cells in antitumor immunity induced by hgp75 and pINGTEM8 DNA vaccines. Mice were not immunized (naive) or immunized with hgp75 DNA vaccine, pINGTEM8 DNA vaccine, or both hgp75 and pINGTEM8 DNA vaccines in mice with or without CD8⁺ T cell depletion.

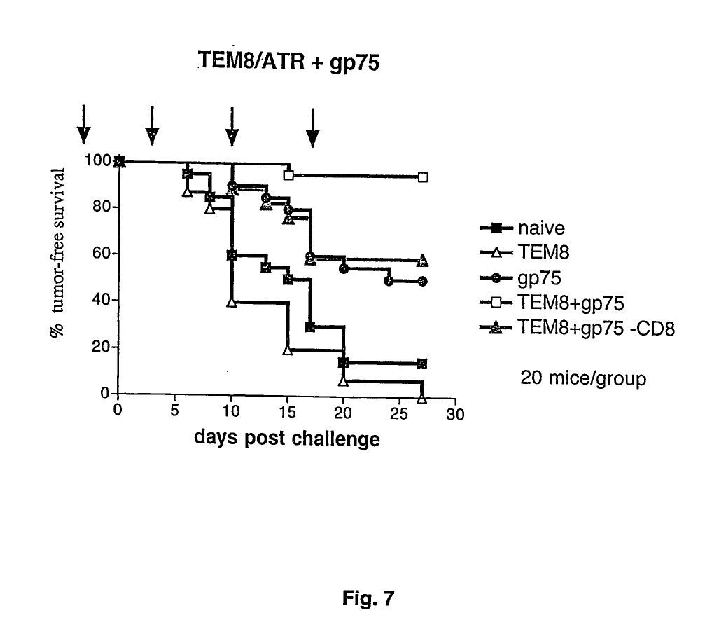

FIG. 8 shows that TEM8 increases tumor immunity in a Surgical Resection Model of B16 Melanoma. Mice were not immunized (naive) or immunized with TRP-2 (tyrosinase-related protein-2), both TRP-2 and pINGTEM8 DNA vaccines or pINGTEM8 DNA vaccine alone.

### DETAILED DESCRIPTION OF THE INVENTION

As used herein, an “antigen” or “immunogen” is a molecule capable of provoking an immune response or to be a target of the elicited immune response.

A “tumor antigen” or “tumor-associated antigen” as used herein is a protein associated with a tumor or a protein expressed within or on the surface of cancer cells and which is capable of provoking an immune response either when presented on the surface of an antigen presenting cell in the context of MHC molecules, or when presented as an intact protein on the cell surface. A tumor-associated antigen may be a shared antigen (i.e. an antigen present also in normal cells), a viral antigen, a differentiation antigen or a mutated antigen capable of triggering a B cell and/or T cell immune response. The antigen can be prepared from cancer cells either by preparing crude extracts of cancer cells, by partially purifying the antigens, by recombinant technology, or by de novo synthesis of known antigens. Tumor-associated antigens include, but are not limited to, peptides, polypeptides, polysaccharides, conjugated polysaccharides, lipids and glycolipids. Tumor cells or tumor cell extracts can also be used as tumor-associated antigen preparations.

A “human at risk of developing cancer” is (i) one exposed to cancer-causing agents such as tobacco, asbestos, or other chemical toxins, viruses or environmental exposure to carcinogens such as radiation; (ii) a subject already treated for cancer and having a low or undetectable tumor burden, but for which a recurrence can be statistically assessed, or (iii) one with a elevated probability of developing cancer on the base of its genetic predisposition, medical condition or prior treatment, viral infection or genetic trait for which a relation to a higher likelihood of developing a cancer has been demonstrated.

A “human having cancer” is a human that has detectable cancerous cells. Cancers or tumors include, but are not limited to, both solid and liquid tumors such as biliary tract cancer, brain cancer, breast cancer, cervical cancer, choriocarcinoma, colon cancer, endometrial cancer, esophageal cancer, gastric cancer, intraepithelial neoplasms, lymphomas, liver cancer, lung cancer (e.g. small cell and non-small cell), melanoma, neuroblastomas, oral cancer, ovarian cancer, pancreas cancer, prostate cancer, rectal cancer, sarcomas, skin cancer, testicular cancer, thyroid cancer, renal cancer, as well as other carcinomas and sarcomas.

Ideal cancer treatments should possess sufficient affinity and specificity to target systemic tumors at multiple sites in the body while discriminating between neoplastic and non-neoplastic cells. In this regard, antigen-specific cancer immunotherapy and immune targeting of tumor neo-vasculature represent two attractive strategies for cancer prevention and treatment. The present invention discloses a new approach that combines these two strategies for antitumor immunity induction resulting in an unexpectedly synergistic response.

Tumor endothelial marker 8 (TEM8) was identified by differential expression screening of endothelial cells in normal and neoplastic human colon (Carson-Walter et al., 2001). TEM8 transcripts are preferentially and abundantly expressed in endothelial calls that line tumor vasculature. The murine counterpart is 96% identical and is highly expressed in the spontaneous mouse melanoma B16 as shown by in situ PCR. The nucleic acid sequence of TEM 8 derived from mouse has SEQ ID No. 1 whereas the nucleic acid sequence of human TEM 8 has SEQ ID No. 4. The murine TEM 8 nucleic acid encodes a 561 amino acid (SEQ ID NO. 2) type 1 transmembrane protein with an I-domain (adhesion motif). On the other hand, the human TEM 8 encodes a 564-amino acid (SEQ ID No. 5) protein. The physiological function of TEM8 is unknown. A portion of the extracellular domain of TEM8 shares high homology with the von Willebrand factor type A domain, which is often found in the extracellular domains of integrins.

Interestingly, an alternative splice product of the TEM8 gene generates the protein anthrax toxin receptor (ATR), which is identical to TEM8 for the first 364 amino acids that include the entire extracellular and transmembrane domains, and then terminates after a 4 amino acid divergence from TEM8. The anthrax toxin receptor protein was identified as an anthrax receptor by isolation and analysis of CHO cells rendered antrax-resistant by mutagenic agents (Bradley et al., 2001).

Because of its expression in tumor vasculature, the investigators examined TEM8 as a target for tumor immunotherapy alone or in conjunction with other tumor-associated antigens. The present invention shows that a DNA vaccine encoding the extracellular domain of TEM8, when used in combination with DNA vaccines encoding differentiation markers expressed in tumors, protect mice from subsequent tumor challenge. Thus, potent antitumor immunity can be generated by combining both a tumor-associated antigen(s) and a TEM8 gene product in one vaccine.

The present invention provides a combination of nucleic acid sequences coding for a tumor-associated antigen(s) and a TEM8 gene product. The nucleic acid sequences can be inserted in suitable expression vectors, such as plasmid or modified virus, which may contain promoters, enhancers, signal or target sequences, all of them suitable for the expression and the subcellular localization of the corresponding polypeptide. Multiple sequences coding for different antigens can be inserted separately or fused together in the same vector. The present invention also provides a composition comprising nucleic acid sequences coding for a tumor-associated antigen(s) and a TEM8 recombinant protein.

In a preferred embodiment of the invention, the active components of the combination are to be used as a vaccine. Principles and methods for vaccine preparation are known to those skilled in the art, as in Paul, “Fundamental Immunology”, Raven Press, New York (1989) or Cryz, S. J., “Immunotherapy and vaccines”, VCH Verlagsgesselshaft (1991). Typically, a DNA vaccine is made of plasmid DNA coding for one or more antigens containing CTL or antibody-inducing epitopes (Wolff et al., 1990). Plasmids can be prepared and used as vaccines according to well-known techniques (Donnelly et al., 1994).

Vaccine compositions usually further contain additives such as emulsifying agent, buffer and adjuvant such as incomplete Freund's adjuvant, aluminum phosphate, aluminum hydroxide or alum. The vaccine composition can also include cytokines such as GM-CSF, IL-2, IL-12, IL-15, IL-18 or CD40L that may further enhance the immune responses.

Formulations containing a combination of tumor antigen(s) and TEM8 gene products, or of either their fragments or derivatives, or of the nucleic acid molecules encoding them, can be used in preventive treatment of subjects at risk of developing cancer. In one embodiment of the invention, the formulation contains HER2 plasmid DNA as the tumor antigen and is used for the prophylactic or therapeutic treatment of breast, uterus, prostate, colon, lung, head and neck cancer. In another embodiment, the formulation contains gp75 plasmid DNA or TRP-2 plasmid DNA as the tumor antigen and is used for the treatment of malignant melanoma Other tumor-associated antigens well-known in the art can be used as tumor antigens in the present invention as well.

Tumor-associated antigen and TEM8 gene product can be encoded by separate DNA molecules. Alternatively, the tumor-associated antigen and TEM8 gene product can be constructed as a fusion protein or a poly-cistronic polypeptide. Moreover, not only can the extracellular domain of TEM8 be incorporated into the vaccine composition, the full length TEM8 sequence can also be used as a vaccine.

Plasmids used as DNA vaccines may be delivered by a variety of parenteral, mucosal and topical routes. For example, the plasmid DNA can be injected by intramuscular, intradermal, subcutaneous (PNAS 83:9551 (1986); WO90/11092) or other routes. It may also be administered by intranasal sprays or drops, rectal suppository or orally. It may also be administered into the epidermis or a mucosal surface using a gene-gun (Johnston, 1992) or Biojector. The plasmids may be given in an aqueous solution, dried onto gold particles or in association with another DNA delivery system including, but not limited to, liposomes, dendramers, cochleate and microencapsulation. It has also recently been discovered that gene-bearing plasmids can be transformed into modified forms of bacteria such as Salmonella which act as delivery vehicles to the immune system. The transformed bacteria can be administered to a host subject orally or by other means well-known in the art.

Nucleic acids such as mRNA can also be loaded onto autologous dendritic cells to be used as vaccine. It is well known in the art that dendritic cells are potent antigen presenting cells, and methods and protocols of inducing immune responses by antigen-loaded dendritic cells are well known in the art.

The tumor antigen vaccines and TEM8 vaccine can be administered simultaneously or separately. Treatment can be started before the diagnosis of tumor, at the appearance of the disease or immediately after surgical removal of the tumor. The administration can be repeated at different time intervals, the doses of the active component of the combination can be varied according to protocols well-known in the art, as long as the patient's condition improves.

In any case, whether the components of the combination are peptides or nucleic acids, they will be in the proper pharmaceutical composition. The pharmaceutical composition will contain effective amounts of both tumor antigen and the TEM8 antigen, wherein the effective amounts are the amounts capable of eliciting a B cell or T cell immune response.

The present invention is directed to compositions of matter useful as a vaccine. The composition contains nucleic acid sequence encoding a tumor-associated antigen (TAA) or a fragment thereof and nucleic acid sequence encoding a tumor endothelial marker 8 (TEM8) or a fragment thereof. Alternatively, the composition contains nucleic acid sequence encoding a tumor-associated antigen or a fragment thereof and a recombinant protein comprising tumor endothelial marker 8 or a fragment thereof. Additionally, the composition also contains recombinant proteins comprising a tumor associated antigen or a fragment thereof and tumor endothelial marker 8 or a fragment thereof. Further, the composition also contains recombinant protein comprising tumor associated antigen or a fragment thereof and nucleic acid sequence encoding tumor endothelial marker 8 or a fragment thereof. Sequences encoding a tumor-associated antigen and the TEM8 antigen can be incorporated into different vectors or into a single vector. Representative examples of tumor-associated antigens include HER2/neu, tyrosinase-related protein 1 (gp75), tyrosinase-related protein 2 (TRP-2) and prostate-specific membrane antigen from any species. In one embodiment, TEM8 protein or a fragment thereof is derived from a mouse or a human. The mouse-derived TEM 8 has nucleic acid sequence of SEQ ID No. 1, which encodes a TEM 8 protein or its fragment having SEQ ID No. 2 or SEQ ID No. 3. The human-derived TEM 8 has nucleic acid sequence of SEQ ID No. 4, which encodes a TEM8 protein or its fragment having SEQ ID No. 5 or SEQ ID No. 3. Additionally, a person having ordinary skill would readily recognize that the TEM8 amino acid sequence may be manipulated to produce a useful TEM8 that is not 100% identical to either SEQ ID NO. 2, SEQ ID No. 3 or SEQ ID No. 5. For example, a person having ordinary skill would find useful a protein that is 80% or 90% homologous to the sequence of SEQ ID NO. 2, SEQ ID No. 3 or SEQ ID No. 5.

The present invention is also directed to methods of using the claimed vaccine compositions to induce antitumor immunity in a subject such as human. In general, the subject is having cancer or at risk of developing cancer. The vector of the vaccine composition can be carried by a delivery vehicle such as liposomes or modified bacteria. When the vaccine composition contains separate vectors, the different vectors can be administered to the subject simultaneously or sequentially. Preferably, the vaccines are administered by intramuscular injection, intradermal injection, subcutaneous injection, intranasal sprays or oral administration. Alternatively, dendritic cells loaded with the vaccines or the proteins encoded by the vaccines can be used to immunize a subject.

The following examples are given for the purpose of illustrating various embodiments of the invention and are not meant to limit the present invention in any fashion. The present examples, along with the methods, procedures, treatments, molecules, and specific compounds described herein are presently representative of preferred embodiments. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### EXAMPLE 1

### Plasmid DNA Construction

pVAXXCDneu was prepared by PCR cloning using pCDneuNT (Invitrogen) cut with HindIII as template and the following primers: sense, 5′-CGCAAGCTTCATCATGGAGCTGGC-3′ (SEQ ID NO:6); antisense,5′-GCAGAATTCTTATGTCACCGGGCT-3′ (SEQ ID NO:7). The PCR conditions were as follows: Step 1, 94° C. for 10 min; Step 2, 95° C. for 1 minute, 58° C. for 1 minute, 72° C. for 2 minutes, 28 cycles; Step 3, 72° C. for 10 min.

After purification of PCR products by Concert Qiagen Kit and further by Qiaex gel extraction kit (Qiagen), the fragment corresponding to extracellular domain of HER2 was cloned into pVAX (Invitrogen, clinical grade) following the steps of: HindIII and EcoRI digestion; T4 DNA overnight ligation; DH5 alfa cells (Takara) transformation by electroporation (BioRad apparatus). The resulting clones were confirmed by sequencing (ABI Prism, Perkin-Elmer).

To construct plasmid pATRex, TEM8 cDNA was prepared from total RNA extracted from mammary tumor arisen in FVB/neuT transgenic mouse. TEM8 cDNA was then PCR-amplified using the following primers: sense-GGACTCTGCGTGGCTGCACTCGTGC (SEQ ID NO:8); antisense-AGAGCAGCGCCAGGGCCAGCAGCAG (SEQ ID NO:9). PCR conditions: Step 1, 95° C. for 5 minutes; Step 2, 95° C. for 1 minute, 64° C. for 1 minute, 72° C. for 2 minutes, 35 cycles; Step 3, 72° C. for 10 minutes. This resulted in the cloning of amino acids 13-278 into pGEM, and this construct was used to generate riboprobes for nucleic acid analysis.

From this plasmid a further extraction was performed in order to clone sequences corresponding to the amino acid sequence 28-278 27-279 of TEM8 (SEQ ID NO:3). The new sequence was PCR cloned with the following primers:

FWKpnIm8 sense-GGGGGTACCGCAATGGGCCGCCGC GAGGATGGGGGA (SEQ ID NO:10); RVEcoRIm8 antisense-GGTGGAATTCCTAGCACAG CAAATAAGTGTCTTC (SEQ ID NO:11). The primers were designed to introduce an ATG translation initiation codon, stop codon and restriction sites for cloning into pcDNA3.1 in order to allow for expression in mammalian cells. PCR conditions: Step 1, 95° C. for 5 min; Step 2, 95° C. for 1 min, 64° C. for 1 min, 72° C. for 2 min, 35 cycles; Step 3, 72° C. for 10 min.

After amplification the fragment was digested with KpnI and EcoRI and cloned into pcDNA3.1. The TEM8 sequence was subsequently excised using EcoRI and KpnI restriction enzymes, and inserted into the EcoRI and KpnI sites of the clinical grade vector pING to create pINGTEM8. The orientation and sequence of TEM8 sequences within pINGTEM8 was confirmed by sequencing.

Primers used for TEM8 recombinant protein: attB1bis-GGGGACAAGTTTGTACAAAAAAGCAGGCTTGATGGGCCGCCGCGAGG ATGGGGGA (SEQ ID NO: 12), attB2bis-GGGGACCACTTTGTACAAGAAAGCTGGGTCGCACAGCAAATAAGTGTC TTC (SEQ ID NO: 13).

Full-length human prostate-specific membrane antigen (PSMA) (Isreali et al., 1993) and mouse PSMA cDNAs (Bacich et al., 1998) were cloned into the clinical grade vector pING (Bergman et al., 2003). The hgp75 DNA has been described (Weber et al., 1998). The method for DNA immunization has been reported (Ross et al., 1997; Weber et al., 1998).

### EXAMPLE 2

### Immunization And Protection Against Mammary Tumor

The therapeutic effectiveness of the invention with regard to eliciting antitumor immunity was tested in a murine model of human mammary cancer. Female FVB mice transgenic for the rat oncogene neu (neuT) under the transcriptional control of the mouse mammary tumor virus promoter/enhancer develop sporadic mammary cancer after sexual maturity. Cells from these “spontaneous” tumors were isolated and cloned to be used for tumor challenge in the same mouse by subcutaneous inoculation. Cancer cells, named VSG A1, isolated from the FVB/neu transgenic mouse were characterized according to FACS analysis for the expression of both neu oncoprotein p185 and MHC Class I molecules. The cells were expanded in a standard medium in presence of 20% FBS. Adherent cells were detached by scraping and controlled for viability immediately before subcutaneous injection in the flank.

DNA immunization was performed by injecting 100 μl sterile saline containing 100 μg of plasmid(s) into the femoral quadriceps. Typically a schedule of three intramuscular injections at two week intervals was used.

The mice were then challenged and monitored for tumor onset by palpation every other day. Tumors were scored as present once they reached a 2 mm diameter and continued to grow. The weight of tumor masses (mg) was calculated by multiplying the square of minor diameter (mm) times the major diameter divided two. Rapidly growing tumor is excised, fixed in formalin and prepared for histological evaluation and vascular immunostaining. In another case the tumor was immediately frozen in liquid nitrogen for TEM8 mRNA analysis. Mice were sacrificed once it was assured the tumors were progressing (usually at a size of about 1 cm).

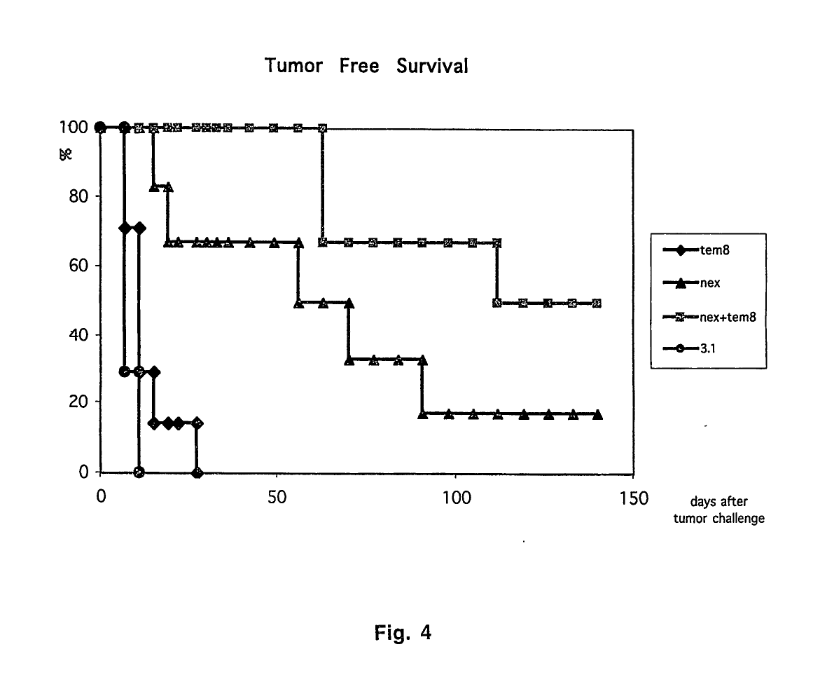

As shown in FIG. 4, mice immunized with pATRex alone showed no protection from tumor growth, whereas immunization with HER2neu gave partial protection. In contrast, immunization with HER2/neu plus pATRex gave nearly complete protection for over 65 days.

### EXAMPLE 3

### Immunization And Protection Against Melanoma

Plasmid DNA was coated on plastic tubing and injected in the skin of mice as described previously (Hawkins et al., 2000). Briefly, abdominal hair of the mice was removed and plasmid DNA was delivered using a helium-driven gun (Accell, PowderJect, Madison, Wis.) into each abdominal quadrant (1 μg plasmid DNA per quadrant). For these experiments, mice (15/group) received pINGTEM8 injections at weekly intervals for 5 weeks. Animals that also received hgp75 injections at the same schedule, but staggered such that hgp75 vaccines were given 3 days after pINGTEM8 vaccines. Animals immunized only with hgp75 received the vaccine for 5 weeks, on the same day that the other mice received the same hgp75 antigen.

Mice were injected intradermally with 3×10⁴ B16 melanoma cells on the right flank 5 days after the final hgp75 DNA immunization. For mice receiving pINGTEM8 only, the challenge fell on day 8 after the final pINGTEM8 vaccine. The mice were then followed for tumor onset by palpation every other day. Tumors were scored as present once they reached a 2 mm diameter and continued to grow. Mice were sacrificed once it was assured the tumors were progressing (usually at a size of about 1 cm).

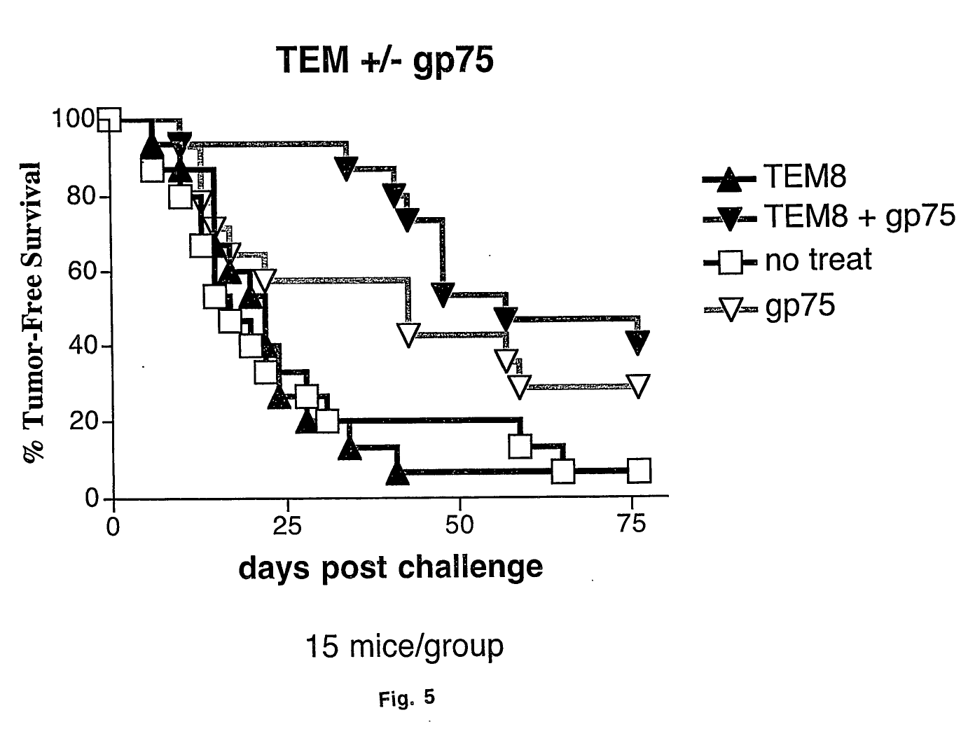

As shown in FIG. 5, TEM8 vaccination alone had no effect on B16 tumor growth. However, on day 40 following tumor challenge, hgp75 immunization alone afforded 57% tumor protection, whereas hgp75+pINGTEM8 immunization resulted in 87% tumor free survival. The antitumor effect is thus synergistic and not additive.

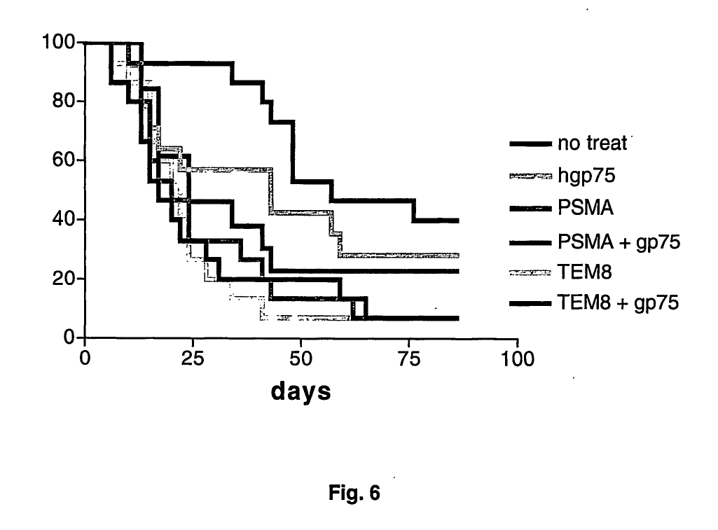

Further, whether the improvement in the immunity induced by immunization with hgp75 and TEM8 DNA vaccines depended on TEM8 was also investigated. In order to accomplish this, the experiment was repeated using the same immunization conditions. However, two additional groups of mice were included in which mice were either immunized with PSMA DNA or both PSMA and hgp75 DNAs. As shown in FIG. 6, TEM8 immunization alone and PSMA immunization alone did not provide tumor protection. Additionally, immunization with both PSMA and hgp75 DNA vaccines did not provide tumor protection either. However, immunization with both TEM8 and hgp75 DNAs provided maximum tumor protection. This demonstrated that immunity induced by hgp75 was dependent on TEM8.

Next, the experiment was repeated using the same immunization conditions, except that an additional group was added included, in which mice immunized with pINGTEM8+hgp75 were depleted of CD8⁺ T cells for 4 weeks starting at day −2 before tumor challenge. As shown in FIG. 6, pINGTEM8 once again showed synergy with the hgp75 vaccine, and this synergy was completely lost in mice lacking CD8⁺ T cells. This strongly supports the idea that pINGTEM8immunity is mediated by CD8⁺ T cell effectors.

### EXAMPLE 4

### Tumor Immunity in a Surgical Resection Model of B16 Melanoma

The therapeutic efficacy of the invention in providing immunity with regard to lung metastasis following resection of a primary tumor challenge was demonstrated using a Surgical Resection Model as described previously (Hawkins et al., 2002). Briefly, mice were injected with B16 melanoma tumor cells into footpad. When the tumor size was 6-8 mm (21-28 days later), the tumor-bearing limb (hind limb) was amputated. The mice were divided into four different treatment groups. One group of mice (n=25) received no treatment (naive). The second group of mice (n=18) was immunized weekly with TRP-2 (tyrosinase-related protein-2) DNA vaccine. The third group of mice (n=22) was immunized weekly with both TRP-2 and TEM8 DNA vaccine. The fourth group of mice (n=25) was immunized weekly with TEM8 DNA vaccine alone. In 21-28 days, the lung mets appear in the sentinels. Hence, the mice were sacrificed 21-28 days later and number of surface lung mets scored.

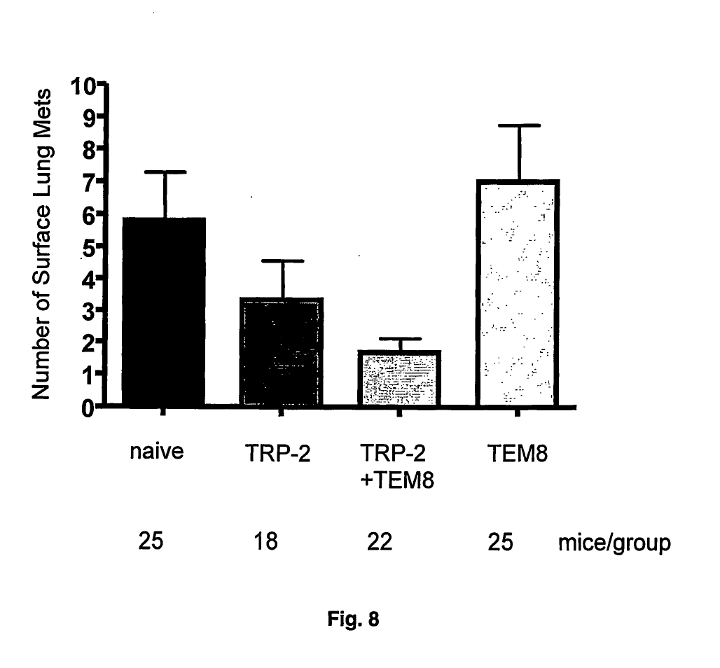

As shown in FIG. 8, immunization with TEM8 DNA alone provided no immunity against the tumor. Although mice immunized with TRP-2 DNA alone showed a decrease in the number of surface lung metastases, the maximum immunity was observed in the group immunized with both TRP-2 and TEM8 DNAs. This demonstrated that TEM8 increased tumor immunity by inhibiting the metastasis of the tumor cells to the lungs.

### EXAMPLE 5

### TEM8 Expression In Prostate Cancer Vasculature

An important consideration in the design of any clinical trial using a vaccine is the determination of the tissue distribution of the antigen, both to support the use of that antigen in a given tumor system, and in highlighting potential sites of harmful autoimmunity. While TEM8 RNA has been detected in the endothelial cells in some human tumors, it has not been characterized in prostate cancer, or in BPH and PIN lesions. TEM8 is expressed in vasculature of PC3 cells grown in nude mice, but this may not reflect the expression pattern in human. Furthermore, the published reports do not provide details of the probes used for the hybridization, and they may not have distinguished between TEM8 and ATR transcripts.

In situ PCR can be used to quantify TEM8 and ATR RNA transcripts in human and mouse prostate tumor sections. Primer pairs specific for the two transcripts can be identified so that TEM8 and ATR may be examined separately. Additional in situ PCR analysis of normal and neoplastic human tissue will allow one to determine where each transcript is expressed. In addition to clinical specimens, mouse models of prostate cancer are available for testing the TEM8 vaccines.

### EXAMPLE 6

### Production of Antibody Specific For TEM8/ATRex

While in situ hybridization or PCR may allow visualization of TEM8 transcripts in the absence of an antibody, it does not provide information on the levels of TEM8 protein expression. Furthermore, more detailed studies on TEM8 post-translation modification, metabolism and interactions with other proteins all require antibody reagents.

In order to characterize vaccines made from full-length TEM8 and identify antibodies that bind native TEM8 on the cell surface, full-length hTEM8 can be cloned and stably expressed in mouse 3T3 cells. A variety of DNA vaccines, including TEM8/ATRex, full-length hTEM8 and TEM8-Flt-3 (a fusion protein of TEM8 and the hemopoietic cytokine Flt3 ligand) can be compared for their ability to induce antibodies to TEM8. Sera will be assayed by Western blots and/or immunohistochemistry, and by flow cytometry using 3T3 cells expressing the full-length TEM8. In this way, it is most likely to identify antibodies with a range of useful specificities. Monoclonal antibodies can be generated using standard techniques.

The following references were cited herein:
Bacich et al., Proc. Am. Assoc. Cancer Res. 39:129 (1998).
Bergmnan et al., Clin. Cancer Res. 9:1284-1290 (2003).
Bradley et al., Nature 414:225-29 (2001).
Carson-Walter et al., Cancer Res. 61:6649-55 (2001).
Donnelly et al., The Immunologist 2:1 (1994).
Guevara et al, Basic Aspects of Tumor Immunlogy, Keystone, Colo., (2003).
Hawkins et al., Surgery 128:273-280 (2000).
Hawkins et al., J Surg Res. 102:137-143 (2002).
Isreali et al., Cancer Res. 53:227-230 (1993).
Johnston, Nature 356:152 (1992).
Ross et al., Clin. Cancer Res. 3:2191-2196 (1997).
Venanzi et al., AACR Annual Meeting, San Francisco, Calif. (2002).
Weber et al., J Clin. Invest. 6:1258-1264 (1998).
Wey et al., Nature Med. 6:1160-66 (2000).
Wolff et al., Science 247:1465-68 (1990).

1. A composition useful as a vaccine, comprising:
(i) one or more vectors comprising a nucleic acid sequence encoding a tumor associated antigen or a fragment thereof or a nucleic acid sequence encoding a tumor endothelial marker 8 or a fragment thereof or a combination thereof; and
(ii) a pharmaceutically acceptable carrier.

2. The composition of claim 1, wherein said tumor-associated antigen is selected from the group consisting of HER2/neu, tyrosinase-related protein 1(gp75), tyrosinase-related protein 2 (TRP-2) and prostate-specific membrane antigen.

3. The composition of claim 1, wherein said nucleic acid sequence encoding a tumor endothelial marker 8 or a fragment thereof is derived from a mouse or a human.

4. The composition of claim 3, wherein said mouse-derived tumor endothelial marker 8 has a nucleic acid sequence of SEQ ID No. 1.

5. The composition of claim 4, wherein said nucleic acid sequence encodes a tumor endothelial marker 8 protein or a fragment thereof having SEQ ID No. 2 or SEQ ID NO. 3.

6. The composition of claim 3, wherein said human-derived tumor endothelial marker 8 has a nucleic acid sequence of SEQ ID. No. 4.

7. The composition of claim 6, wherein said nucleic acid sequence encodes a tumor endothelial marker 8 protein or a fragment thereof having SEQ ID No. 5 or SEQ ID No. 3.

8. The composition of claim 3, wherein said tumor endothelial marker 8 has an amino acid sequence 80% homologous to SEQ ID No. 2, SEQ ID No. 3 or SEQ ID No. 5.

9. The composition of claim 3, wherein said tumor endothelial marker 8 has an amino acid sequence 90% homologous to SEQ ID No.2, SEQ ID No. 3 or SEQ ID No. 5.

10. The composition of claim 1, wherein said vector is a plasmid.

11-20. (canceled)

21. A method of inducing antitumor immune responses in an individual, comprising the step of administering to said individual the composition of claim 1.

22. The method of claim 21, wherein said composition is administered by a means selected from the group consisting of intramuscular injection, intradermal injection, subcutaneous injection, intranasal sprays and oral administration.

23. The method of claim 21, wherein said vectors of said composition are administered to said individual simultaneously or sequentially.

24. The method of claim 21, wherein the vectors of said composition are carried by delivery vehicles selected from the group consisting of liposomes and bacteria.

25. The method of claim 21, wherein said individual has been diagnosed as having cancer or is at risk of developing cancer.

26. A method of inducing antitumor immune responses in an individual, comprising the step of administering to said individual dendritic cells comprising nucleic acid or protein selected from the group consisting of the composition of claim 1 and proteins encoded by the vectors of said composition.

27. The method of claim 26, wherein said individual has been diagnosed as having cancer or is at risk of developing cancer.

28-33. (canceled)

34. A composition useful as a vaccine, comprising:
a. a vector comprising a nucleic acid sequence encoding a tumor-associated antigen or a fragment thereof and a recombinant protein comprising tumor endothelial marker 8 or a fragment thereof or a vector comprising a nucleic acid sequence encoding a tumor endothelial marker 8 or a fragment thereof and a recombinant protein comprising a tumor associated antigen or a fragment thereof or a recombinant protein comprising tumor endothelial marker 8 or a fragment thereof and a recombinant protein comprising a tumor associated antigen or a fragment thereof; and
b. a pharmaceutically acceptable carrier.

35. The composition of claim 34, wherein said tumor-associated antigen is selected from the group consisting of HER2/neu, tyrosinase-related protein 1 (gp75), tyrosinase-related protein 2 (TRP-2) and prostate-specific membrane antigen.

36. The composition of claim 34, wherein said recombinant protein comprising tumor endothelial marker 8 or a fragment thereof is derived from a mouse or a human.

37. The composition of claim 36, wherein said mouse-derived tumor endothelial marker 8 protein or fragment thereof has an amino acid sequence of SEQ ID No. 2 or SEQ ID No. 3.

38. The composition of claim 37, wherein said amino acid is encoded by nucleic acid of SEQ ID No. 1.

39. The composition of claim 36, wherein said human-derived endothelial marker 8 protein or a fragment thereof has an amino acid sequence of SEQ ID No. 5 or SEQ ID No. 3.

40. The composition of claim 39, wherein said amino acid is encoded by nucleic acid of SEQ ID No. 4.

41. The composition of claim 36, wherein said tumor endothelial marker 8 has an amino acid sequence 80% homologous to SEQ ID No. 2, SEQ ID No. 3 or SEQ ID No. 5.

42. The composition of claim 36, wherein said tumor endothelial marker 8 has an amino acid sequence 90% homologous to SEQ ID No. 2, SEQ ID No. 3 or SEQ ID No. 5.

43. The composition of claim 34, wherein said vector is a plasmid.

44. A method of inducing antitumor immune responses in an individual, comprising the step of administering to said individual the composition of claim 34.

45. The method of claim 44, wherein said composition is administered by a means selected from the group consisting of intramuscular injection, intradermal injection, subcutaneous injection, intranasal sprays and oral administration.

46. The method of claim 44, wherein said vectors of said composition and the recombinant proteins of said composition are administered to said individual simultaneously or sequentially.

47. The method of claim 44, wherein the vector of said composition is carried in a delivery vehicle selected from the group consisting of liposomes and bacteria.

48. The method of claim 44, wherein said individual is diagnosed as having cancer or is at risk of developing cancer.

49-76. (canceled)
